(19) United States
(12) Patent Application Publication
(10) Pub. No.: US 2026/0315823 A1
(43) Pub. Date: Oct. 15, 2026

(54) SAUNA APPARATUS

(71) Applicant: JTL Enterprises, Inc., Clearwater, FL (US)

(72) Inventors: Paul LUNTER, Palm Harbor, FL (US); Kevin CONAWAY, Tampa, FL (US); William DASKAM, New Port Richey, FL (US); Nathan BARROW, Clearwater, FL (US); Timothy ELLIOTT, Pinellas Park, FL (US)

(21) Appl. No.: 19/466,873

(22) Filed: Feb. 2, 2026

Related U.S. Application Data
(60) Provisional application No. 63/753,278, filed on Feb. 3, 2025.

(51) Int. Cl. A61N 5/06; H05B 3/00; A61N 5/00

(52) U.S. Cl. CPC A61N 5/0625; H05B 3/0085; A61N 2005/007; A61N 2005/0626; A61N 2005/0636; A61N 2005/0659; A61N 2005/0663; H05B 2203/032

(57) ABSTRACT

A sauna apparatus comprising a housing defining an interior space in which a user support is located. A door openable to allow access to the interior space is also provided. A plurality of infrared heat sources are located in the interior space. A plurality of light therapy sources are also located in the interior space. A controller is operative to control operation of the infrared heat sources and the light therapy sources.

### CROSS-REFERENCE TO A RELATED APPLICATION

This application is based upon and claims the benefit of U.S. provisional patent application serial no. 63/753278, filed Feb. 3, 2025, which is incorporated fully herein by reference for all purposes.

### FIELD OF THE INVENTION

The present invention relates generally to saunas, particularly saunas that might be used in a spa or commercial setting.

### BACKGROUND OF THE INVENTION

The origins of saunas reportedly date back hundreds of years, with many different cultures around the world incorporating this practice. According to the Cleveland Clinic, saunas reportedly may be used for pain relief, stress relief, improved cardiovascular health, and a variety of other potential benefits. https://health.clevelandclinic.org/sauna-benefits. The popularity and demand for saunas has increased in recent years, with one media outlet reporting that “Google UK searches for ‘home sauna’ rose by 84% between January and March 2024 and the same period in 2020, according to the marketing company Semrush.” Traditional forms of saunas include steam, wood-burning, or electrically heated options, whereas infrared heat saunas have emerged recently as a more modern solution.

Traditionally, saunas have utilized various forms of wood construction, as wood is a material that stays relatively cool to touch in warmer environments and also provides a nice aesthetic appeal. While this is a very good solution for in-home low-usage applications, wood construction inherently has some disadvantages in larger-scale, commercial settings. Throughout each session, the heat produced inside a sauna naturally causes users to sweat. Depending on the amount of sweat produced, this leads to both safety and sanitary concerns from one user to the next, as the sweat and moisture tends to pool. Even after wiping down wet surfaces, the wood may absorb moisture and may not completely dry prior to the next session. Also, with wood being a natural material, it may require the owner to refinish or replace the wood surfaces periodically after repeated exposure to sweat, dirt, and grime.

The present invention recognizes and addresses considerations of prior art constructions and methods.

### SUMMARY OF THE INVENTION

One aspect of the present invention provides a sauna apparatus comprising a housing defining an interior space in which a user support is located. A door openable to allow access to the interior space is also provided. A plurality of infrared heat sources are located in the interior space. A plurality of light therapy sources are also located in the interior space. A controller (e.g., inside the housing) is operative to control operation of the infrared heat sources and the light therapy sources.

Some exemplary embodiments comprise a user interface mounted in the interior space.

According to some exemplary embodiments, a forced air circulation system is operative to circulate air through the interior space of the housing. For example, the forced air circulation system may be configured to move air across a back of the infrared heat sources.

According to some exemplary embodiments, the infrared heat sources may include far infrared panels and at least one of quartz heaters and graphene heaters.

According to some exemplary embodiments, the light therapy sources emit light at one or more wavelengths falling in a range of 600-1000 nm. For example, the light therapy sources may emit light at a first wavelength and a second wavelength. The first wavelength may fall in a range of approximately 600-700 nm and the second wavelength may fall in a range of approximately 800-900 nm.

According to some exemplary embodiments, surfaces in the interior space may be formed of a composite material having a polymeric component.

According to some exemplary embodiments, flow channels may be defined to conduct water collecting in the interior space out of the interior space.

According to some exemplary embodiments, an air filtration system may be provided.

According to some exemplary embodiments, at least one cooling fan may be provided.

According to some exemplary embodiments, at least one blower, operative to at least partially dry a user, may be provided.

According to some exemplary embodiments, the user support may be in the form of a seat.

Another aspect of the present invention provides a sauna apparatus comprising a housing defining an interior space in which a user support is located. A door openable to allow access to the interior space is also provided. A plurality of infrared heat sources are located in the interior space. A controller is operative to control operation of the infrared heat sources and a user interface is mounted in the interior space. A forced air circulation system is operative to circulate air through the interior space of the housing.

A further aspect of the present invention provides a sauna apparatus comprising a housing defining an interior space in which a user support is located. A door openable to allow access to the interior space is also provided. A plurality of infrared heat sources are located in the interior space. A plurality of flow channels are defined to conduct water collecting in the interior space out of the interior space to a drain pan. A drain pan blower is operative to force air over condensate that has collected in the drain pan.

The accompanying drawings, which are incorporated in and constitute a part of this specification, illustrate one or more embodiments of the disclosure and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended drawings, in which;

FIG. 1 is a perspective view of a sauna apparatus in accordance with an embodiment of the present invention.

FIG. 2 shows the sauna apparatus of FIG. 1 in use.

FIG. 3 is an enlarged view of a portion of FIG. 2.

FIG. 4 is a perspective view of the sauna apparatus of FIG. 1 with the door open to show various internal components.

FIGS. 5 and 6 are enlarged views showing certain internal components of the sauna apparatus of FIG. 1.

FIG. 7 shows certain aspects of the sauna apparatus of FIG. 1 in use as well as the position of air intakes for an air circulation system.

FIGS. 8 and 9A-9B diagrammatically illustrate aspects of the operation of the air circulation system.

FIGS. 10A and 10B diagrammatically illustrate various components of the sauna apparatus of FIG. 1.

FIG. 11 diagrammatically illustrates operation of a drain pan blower of the sauna apparatus of FIG. 1.

FIG. 12 diagrammatically illustrates operation of the air circulation system of the sauna apparatus of FIG. 1.

Repeat use of reference characters in the present specification and drawings is intended to represent same or analogous features or elements of the invention according to the disclosure.

### DETAILED DESCRIPTION OF PREFERRED EMBODIMENTS

Reference will now be made in detail to presently preferred embodiments of the invention, one or more examples of which are illustrated in the accompanying drawings. Each example is provided by way of explanation, not limitation, of the invention. In fact, it will be apparent to those skilled in the art that modifications and variations can be made in the present invention without departing from the scope and spirit thereof. For instance, features illustrated or described as part of one embodiment may be used on another embodiment to yield a still further embodiment. Thus, it is intended that the present invention covers such modifications and variations as come within the scope of the appended claims and their equivalents.

For any commercial business such as health clubs and spas that provide either red light therapy or saunas, embodiments of the present invention provide a more innovative, commercial grade and scalable solution, fusing both red light and infrared sauna technology. Preferably, multiple wavelengths of light therapy are provided. In addition, commercial settings often contribute to the presence of heavy amounts of moisture, wetness, or sweat. Embodiments of the present invention may be sprayed or completely wiped down, sanitized, and dried between sessions. A blower/fan may be installed (e.g., on an interior wall of the device) to dry off the user after a session. These features provide a cleaner, drier solution which minimizes any sanitary or slip-and-fall risks for the user or others walking by the device.

The sauna is preferably constructed with commercial-grade composite material for limited maintenance, as opposed to the construction of traditional wood saunas. The surfaces do not absorb sweat or moisture (unlike traditional wood sauna surfaces which do absorb moisture) and may be easily wiped down and sanitized between sessions to provide a clean, dry surface for the next user, free of bacteria or germs.

In a preferred embodiment, the user sits in a semi-reclined position during the session, with the back wall and seat both angled slightly for comfort, and the device is designed for use by one person at a time. As noted, commercial grade flooring and seating is included, combined with channels to trap and eliminate excess moisture or perspiration caused by the heat of the sauna. This also provides a means to remove water should the user enter the sauna wet (e.g., if the user came from the pool or cold plunge). Preferably, multiple forms of air filtration systems are also included to help keep the air clean and reduce or eliminate any odor or germs.

During the session, the user has access to multiple forms of heat and light therapy, as the device preferably includes at least two or more different forms of infrared heat (e.g., far infrared panels and near infrared heater tubes) and at least two or more wavelengths of light therapy (600-700nm for skin rejuvenation and 800-900nm for recovery). The heat and light therapy sources are positioned strategically in various locations inside the device, e.g., including the back, both sides of face, hands, knees, and shins (both sides), chest and neck, etc.

Preferably, the user may activate and control various features of the device with a touchscreen interface located inside the sauna so as to be convenient for the user. The sauna preferably has a special cooling system so the touchscreen’s processor stays cool and will not fail from heat exposure. The owner of the sauna may control access to the device with an optional external timer feature. Other features may include scent solutions, speakers for relaxation music, and salt therapy bricks to enhance the session.

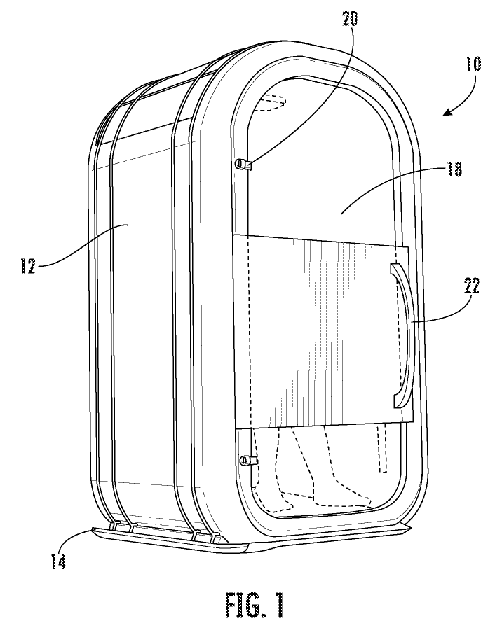

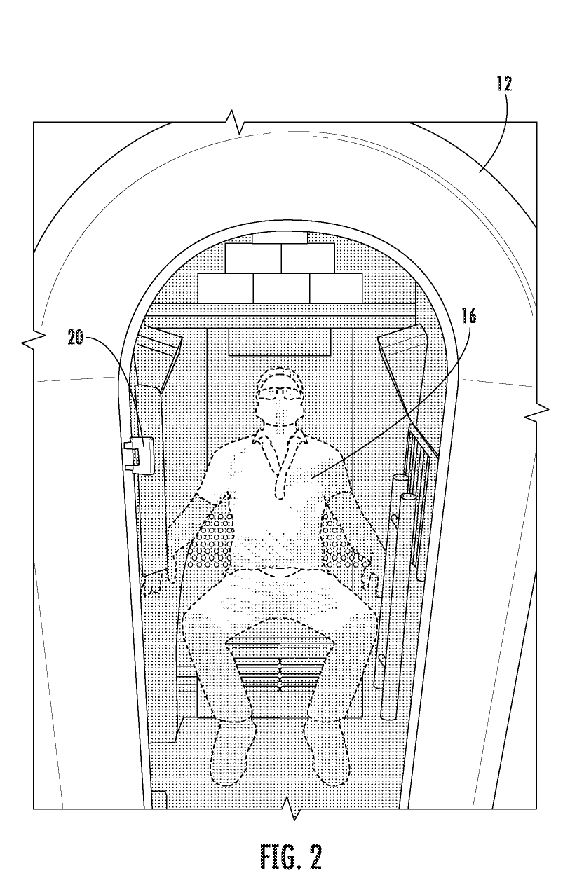

FIGS. 1-3 illustrate a sauna apparatus 10 constructed in accordance with an embodiment of the present invention. As shown, apparatus 10 includes an upstanding housing 12 supported on a floor via a pedestal portion 14. The housing 12 defines an interior space in which a user 16 will sit during the session (e.g., in a semi-reclined position). In this case, apparatus 10 is designed to have only one occupant at a time. The interior space is accessed by a door 18, which in this case is at least semi-transparent. For example, the door 18 may be formed of tempered glass or a similar material suitable for use in this application. In addition, the door 18 may have a covering or etched region (as shown) from approximately the shins to the neck for privacy but still allowing the user the ability to see people walking by or up to the unit. The door 18 is mounted to the housing 12 via suitable hinges (e.g., hinge 20). A door handle 22 may also be provided adjacent to the side of the door opposite the hinges.

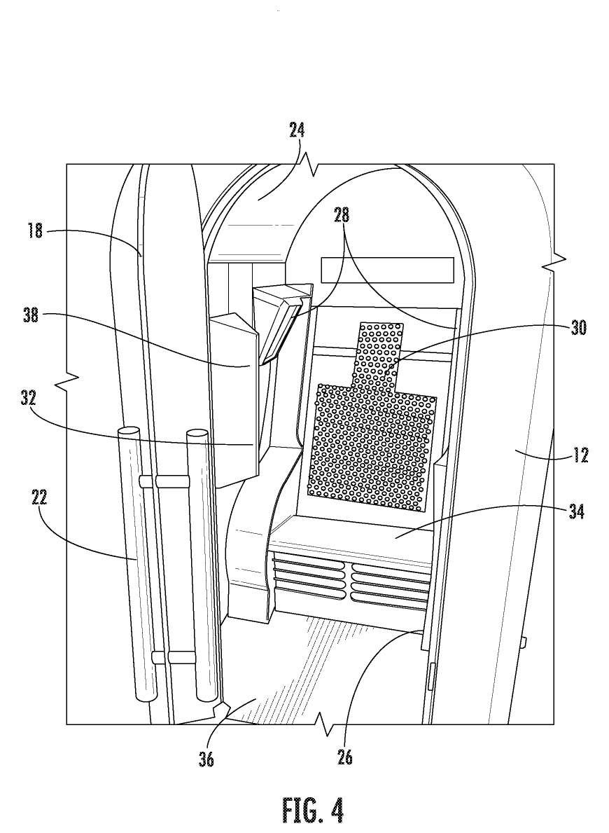

Referring now also to FIGS. 4-6, various infrared energy and red-light sources are mounted inside the housing 12 so that the energy will impinge on the user’s body during the session. In this regard, multiple forms of infrared heat sources (e.g., above and in front of the user), may preferably be provided, as indicated, for example, at 24 and 26. In preferred embodiments, these may include far infrared panels and quartz (or graphene) heaters to provide sufficient heat for a range of temperature 100 degrees F and higher.

In addition, multiple light therapy energy sources may also be provided. For example, as indicated at 28, 30, and 32, the light therapy sources may take the form of light panels behind, left top, right top, and in front of the user. The light panels may emit light energy at multiple wavelengths over the range of 600-1000 nm. For example, in a preferred embodiment, first sources may emit light energy at 630 nm while second sources emit light energy at 850 nm. Each of these wavelengths is known to provide different therapeutic benefits to the user. One skilled in the art will appreciate that each of the light panels may be constructed of a plurality of individual light emitters (e.g., LEDs) with some emitters configured to produce the first wavelength while other emitters produce the second wavelength. The red light radiance and irradiance energy levels are preferably automatically adjusted based on the length of time the user chooses at the beginning of the session, so every user gets the same benefits.

As noted above, various surfaces of sauna apparatus 10, such as the seat 34, flooring 36, and the interior walls, are preferably made of suitable composite materials that do not absorb water and are easily cleaned. In addition, the materials are preferably selected to handle operation temperatures of the sauna (up to about 140˚ F) while also having an effusivity that does not become uncomfortably hot to touch during use. One of the advantages of wood used in prior art saunas is its low effusivity (when compared, for example, to more effusive materials such as metal). Here, the composite materials that are selected may advantageously have an effusivity similar to wood (less than 2.0). Preferred materials, for example, may have an effusivity of about 1.9. Suitable polymeric materials for this purpose include, but are not limited to HDPE and ABS.

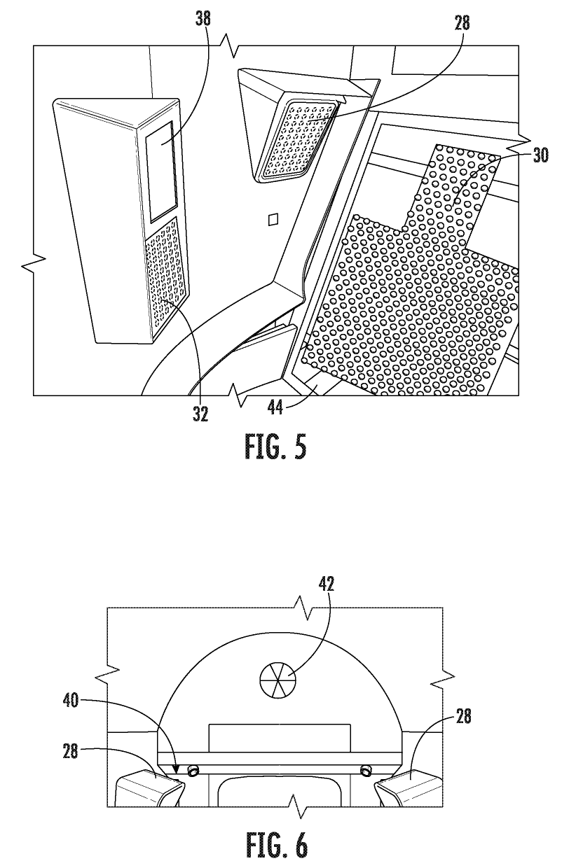

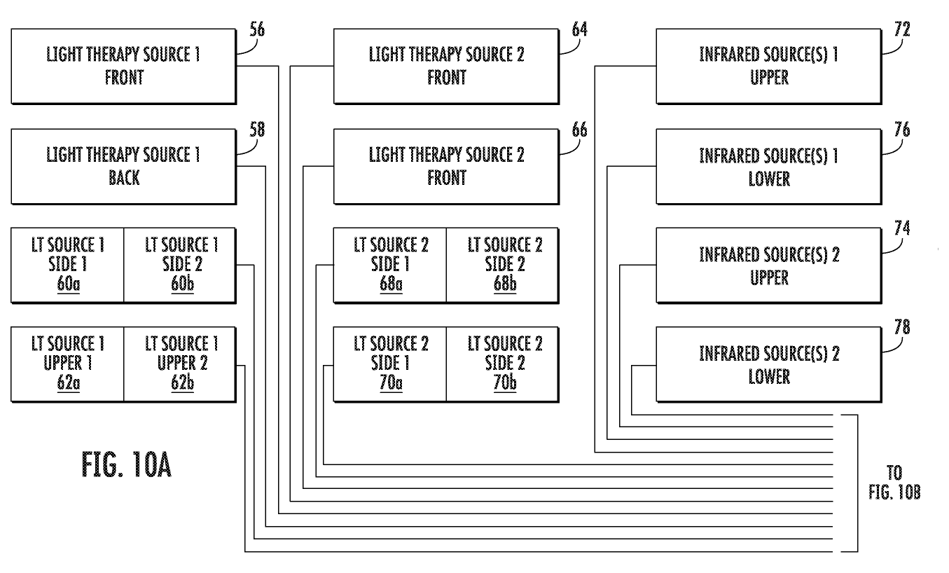

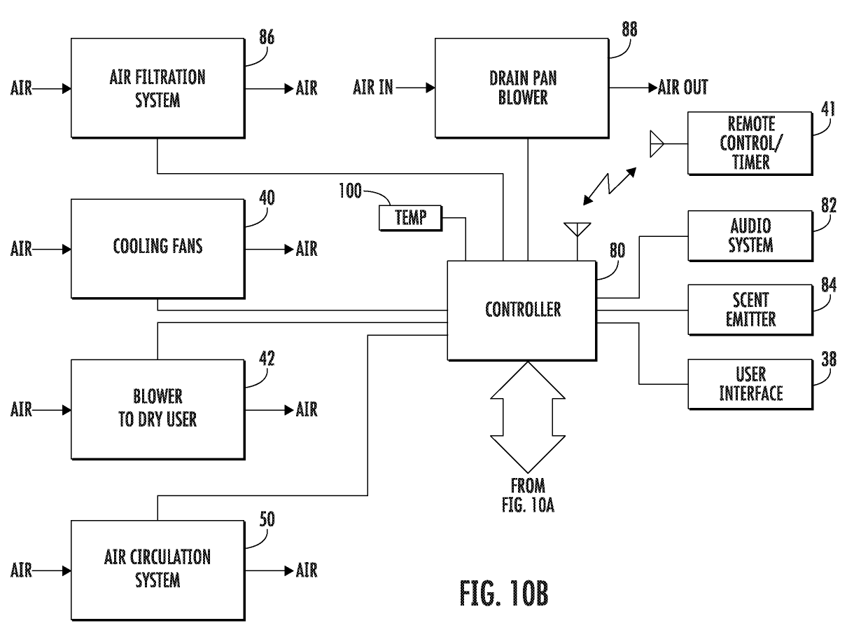

Referring specifically to FIG. 5, a suitable user interface is preferably provided inside of sauna apparatus 10 so that the user can control functions of the unit’s operation. Preferably, the user interface may take the form of a touchscreen display 38. Selectable aspects may include temperature, light wavelength, which light panels are on (i.e., so that the area of the body receiving light therapy can be controlled, etc.). Also, as indicated in FIG. 6, at least one cooling fan 40 for comfort may also be operated from the user interface 38. (Preferred embodiments utilize a pair of cooling fans 40 in the back upper corners of the interior space. Various aspects may also be controlled remotely, such as by an external timer 41 (FIG. 10B).

A blower fan 42 for drying off the user is also preferably provided. As shown, blower fan 42 is installed in this embodiment on an interior wall of the unit. Preferably, the blower fan will be activated automatically after the session to help remove excess sweat/moisture from the user. This will also reduce the risk of slip and fall, as the user is less likely to walk around dripping sweat.

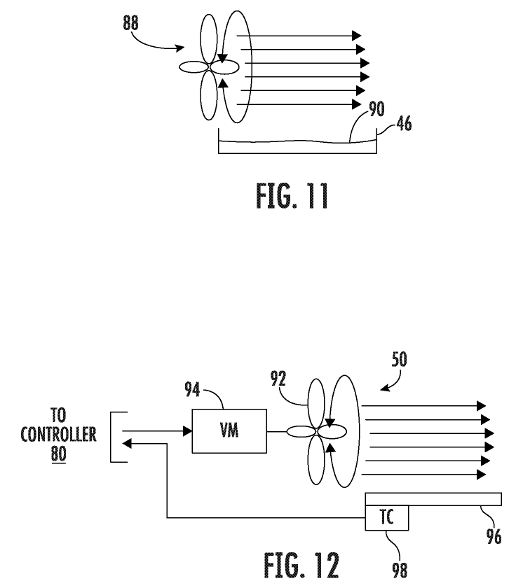

Sauna apparatus 10 is further configured so that any moisture that collects in the interior compartment in which the user sits will drain away. In this regard, the interior surfaces of the compartment are preferably configured so that the moisture will be conducted into one or more channels 44 at the bottom of the housing 12. In this embodiment, collected water flows along the channels into a drain pan 46 (FIG. 11) at the bottom of the housing 12.

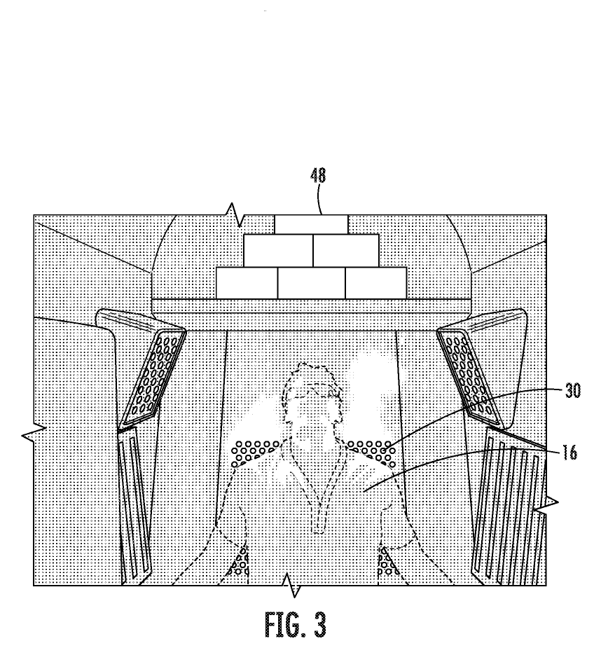

As most easily seen in FIG. 3, sauna apparatus 10 may be adapted to provide salt therapy during a user session. In this regard, apparatus 10 has a plurality of salt bricks 48 that release some salt into the air inside of housing 12. Salt therapy is believed to provide various benefits to the user, especially benefits relating to breathing.

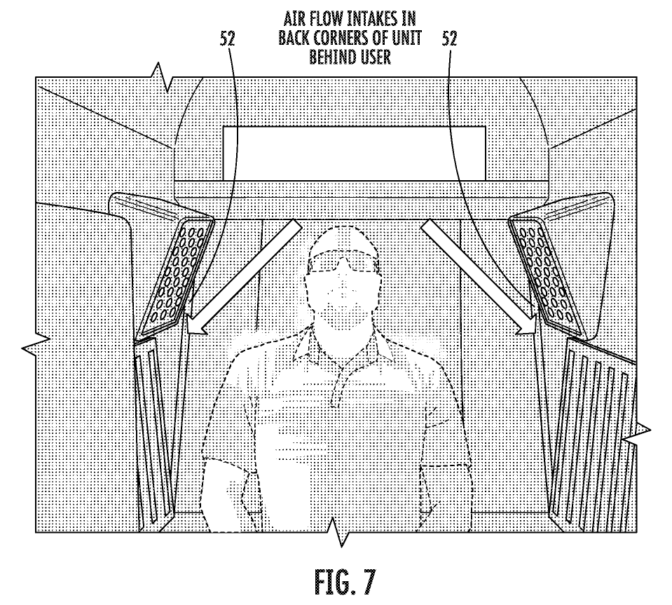

Certain additional aspects of sauna apparatus 10 will now be described with particular reference to FIGS. 7, 8, and 9A-9B. In this regard, sauna apparatus 10 preferably includes a forced air circulation system (diagrammatically indicated at 50 in FIG. 10B) that regulates the temperature more evenly throughout the unit. The air circulation system 50 keeps the heaters running efficiently, eliminates hot spots, and ensures that surfaces, including the heaters themselves, can be safely touched by the user. As will be explained below, the air circulation system preferably operates variably, drawing heat away from the back side of the heaters, keeping them below a temperature that is uncomfortable to the touch. It will be appreciated that the air circulation system may also advantageously extend the operational life of the infrared sources (“heaters”) because they will not be constantly operated at an elevated temperature.

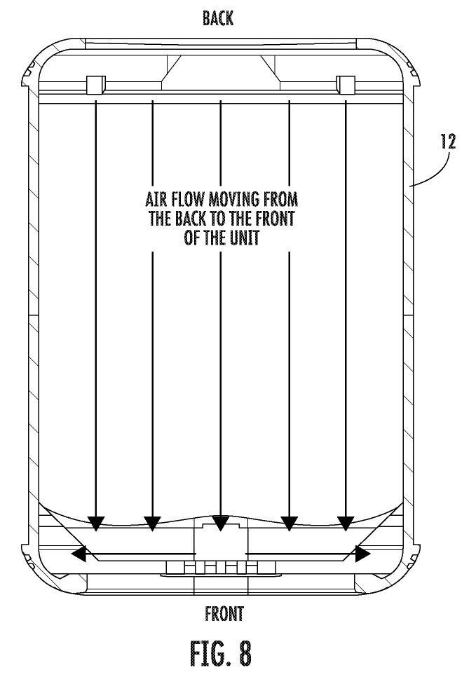

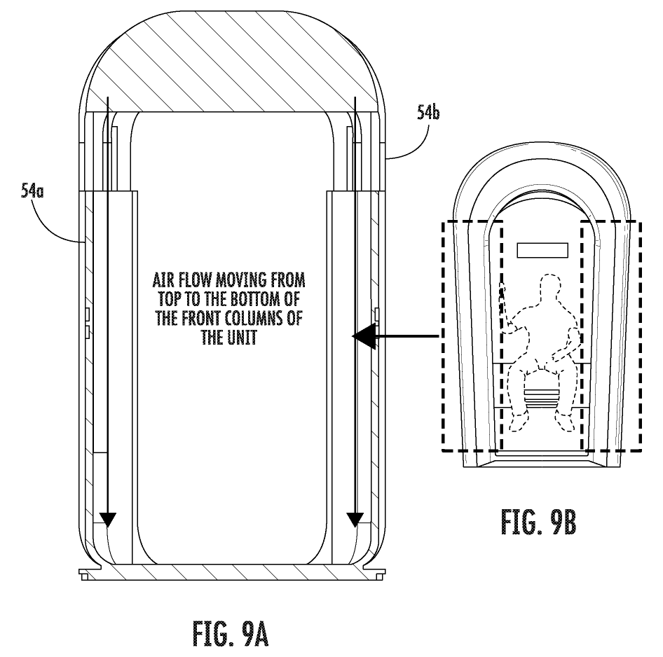

As shown in FIG. 7, the air is preferably drawn in through several air flow intakes 52 provided in the user compartment. As shown in FIG. 8, the air then passes from back to front in a plenum defined at the top of housing 12. Specifically, from the intakes 52, air is ducted up to the top of the back of the unit, and then pushed to the front of the unit, passing along the ceiling of the unit, behind the far infrared heater panels. Next, as shown in FIG. 9A, the air flows down through plenums defined in the left and right vertical columns 54a-b of the housing 12. In doing so, the air passes over the near infrared quartz heaters, before emptying back into the interior space of sauna apparatus 10 through outlets near the bottom of the columns. The recirculation is variable but continuous, as indicated in FIG. 9B.

FIGS. 10A and 10B diagrammatically illustrate various components of sauna apparatus 10. Referring first to FIG. 10A, first light therapy sources (e.g., red light of a first wavelength) are preferably positioned in the interior space of the housing 12 so as to illuminate the following areas of user’s body: front (source 56), back (source 58), sides (sources 60a-b), and upper body (sources 62a-b). Similarly, second light therapy sources (e.g., red light of a second wavelength) are preferably positioned in the interior space of the housing 12 so as to illuminate the following areas of user’s body: front (source 64), back (source 66), sides (sources 68a-b), and upper body (sources 70a-b). Additionally, upper infrared sources of first and second types (infrared panels and quartz (or graphene) heaters) are preferably provided (sources 72 and 74). Lower infrared sources of the first and second types are also preferably provided (sources 76 and 78).

Referring now to FIG. 10B, the heater and light therapy sources are controlled by a “controller” 80, which may take the form of one or more suitable processing chips, whether referred to as processors, microprocessors, controllers, microcontrollers, processing units, etc. “Controller” may also include circuitry ancillary to the processing chip(s) such as input-output circuitry, digital-to-analog converters, current loop circuitry, etc. as necessary or desired. The heat sources and software are preferably modulated to maximize the heat potential in the shortest time to raise the cabin temperature twenty or more degrees without the heating sources getting warm to touch.

The controller 80 is in electrical communication with the user interface 38 so that the user can select various functions of the device’s operation. These may include content to be played over an integral audio system 82 (such as soothing music). Preferred embodiments of sauna apparatus 10 may also include a controllable scent emitter 84 to effuse a desired scent into the interior space of housing 12 during a session. A remote control device, e.g., timer 41, may wirelessly communicate with controller 80, as shown.

Multiple air circulation and/or air movement systems are also operated by controller 50. These include cooling fans 40 and blower 42. An air filtration system 86, preferably including multiple types of filtration elements (e.g., HEPA, charcoal, etc.), may also be provided to keep the interior air clean and/or reduce germ count. A drain pan blower 88 may also be provided. As shown in FIG. 11, drain pan blower 88, in this embodiment, forces outside air over the condensate 90 that has collected in drain pan 46 to accelerate evaporation of the condensate. The resulting vapor, now entrained in the flowing air, is then exhausted to ambient (i.e., outside of sauna apparatus 10).

As noted above, sauna apparatus 10 includes an air circulation system 50 providing a forced airflow path that balances the temperature inside the unit, ensuring all space inside the sauna is heated to substantially the same temperature (avoiding the top of the interior space being too hot and the bottom of the interior space not being hot enough). The heaters (i.e., the infrared heating units) are kept cool enough to be touch safe during the session by circulation of air across the back of the heaters. System 50 provides a safe and efficient way to heat the sauna, as the heaters remain on the entire session and maintain a constant temperature.

Certain aspects of air circulation system 50 may be most easily explained with reference to FIG. 12. As shown, a fan 92 is in this case driven by a variable speed motor 94. The infrared heaters, such as source 96, are each equipped with a suitable temperature sensor, such as thermocouple 98, which provides an indication of the heaters’ temperature to controller 80. Based on this information and the indication of air temperature in the interior space of the sauna apparatus 10, e.g., as indicated by an air temperature sensor 100, the controller 80 varies the speed of the fan 92. The air flow path forces air across the back of the heaters to draw away heat that is generated by their use. The user interface may be similarly cooled by system 50 or a separate forced air system may be used for this purpose. The user interface (touchscreen) may preferably be equipped with heat sink fins on its back surface to facilitate heat dissipation.

It can thus be seen that the present invention provides a novel sauna apparatus. While one or more preferred embodiments of the invention are described above, it should be appreciated by those skilled in the art that various modifications and variations can be made in the present invention without departing from the scope and spirit thereof.

What is claimed is:

1. A sauna apparatus comprising:
a housing defining an interior space in which a user support is located;
a door openable to allow access to the interior space;
a plurality of infrared heat sources located in the interior space;
a plurality of light therapy sources located in the interior space; and
a controller operative to control operation of the infrared heat sources and the light therapy sources.

2. A sauna apparatus as set forth in claim 1, further comprising a user interface mounted in the interior space.

3. A sauna apparatus as set forth in claim 1, further comprising a forced air circulation system operative to circulate air through the interior space of the housing.

4. A sauna apparatus as set forth in claim 3, wherein the forced air circulation system is configured to move air across a back of the infrared heat sources.

5. A sauna apparatus as set forth in claim 1, wherein the infrared heat sources include far infrared panels and at least one of quartz heaters and graphene heaters.

6. A sauna apparatus as set forth in claim 1, wherein the light therapy sources emit light at one or more wavelengths falling in a range of 600-1000 nm.

7. A sauna apparatus as set forth in claim 6, wherein the light therapy sources emit light at a first wavelength and a second wavelength.

8. A sauna apparatus as set forth in claim 1, wherein the first wavelength falls in a range of approximately 600-700 nm and the second wavelength falls in a range of approximately 800-900 nm.

9. A sauna apparatus as set forth in claim 1, wherein surfaces in the interior space are formed of a composite material having a polymeric component.

10. A sauna apparatus as set forth in claim 1, wherein flow channels are defined to conduct water collecting in the interior space out of the interior space.

11. A sauna apparatus as set forth in claim 1, further comprising an air filtration system.

12. A sauna apparatus as set forth in claim 1, further comprising at least one cooling fan.

13. A sauna apparatus as set forth in claim 1, further comprising at least one blower operative to at least partially dry a user.

14. A sauna apparatus as set forth in claim 1, user support is in the form of a seat.

15. A sauna apparatus comprising:
a housing defining an interior space in which a user support is located;
a door openable to allow access to the interior space;
a plurality of infrared heat sources located in the interior space;
a controller operative to control operation of the infrared heat sources;
a user interface mounted in the interior space; and
a forced air circulation system operative to circulate air through the interior space of the housing.

16. A sauna apparatus as set forth in claim 15, wherein the forced air circulation system is configured to move air across a back of the infrared heat sources.

17. A sauna apparatus as set forth in claim 15, wherein the infrared heat sources include far infrared panels and at least one of quartz heaters and graphene heaters.

18. A sauna apparatus as set forth in claim 15, further comprising a plurality of light therapy sources located in the interior space, the light therapy sources operative to emit light at one or more wavelengths falling in a range of 600-1000 nm.

19. A sauna apparatus as set forth in claim 15, wherein surfaces in the interior space are formed of a composite material having a polymeric component.

20. A sauna apparatus comprising:
a housing defining an interior space in which a user support is located;
a door openable to allow access to the interior space;
a plurality of infrared heat sources located in the interior space;
a drain pan;
a plurality of flow channels defined to conduct water collecting in the interior space out of the interior space to the drain pan; and
a drain pan blower operative to force air over condensate that has collected in the drain pan.
